(19) United States
(12) Patent Application Publication
(10) Pub. No.: US 2020/0350079 A1
(43) Pub. Date: Nov. 5, 2020

(54) Epidemic Alert System

(71) Applicants: James Thomas, Whiting, IN (US); Yanette B. Munoz, Hammond, IN (US)

(72) Inventors: James Thomas, Whiting, IN (US); Yanette B. Munoz, Hammond, IN (US)

(21) Appl. No.: 16/863,598

(22) Filed: Apr. 30, 2020

Related U.S. Application Data
(60) Provisional application No. 62/841,574, filed on May 1, 2019.

(51) Int. Cl. G16H 50/80; G16H 10/60; G16H 50/20; G16H 20/10; G06Q 50/14; G06Q 50/26; G06Q 10/10; G01C 21/20

(52) U.S. Cl. CPC G16H 50/80; G16H 10/60; G16H 50/20; G01C 21/20; G06Q 50/14; G06Q 50/265; G06Q 10/1093; G16H 20/10

(57) ABSTRACT

The present disclosure provides a method for providing vaccination information. The method includes the steps of receiving vaccination records about a person, receiving travel information about the person including a destination of travel, receiving infectious outbreak information about the destination, determining vaccination need and/or timing information based on the travel information and the vaccination records of the person, and transmitting the vaccination need and/or timing information to the person.

### CROSS-REFERENCE TO RELATED APPLICATIONS

The application claims the benefit of U.S. Provisional Application No. 62/841,574, filed on May 1, 2019, and entitled “EPIDEMIC ALERT SYSTEM,” the complete disclosure of which is expressly incorporated by reference herein.

### FIELD OF THE INVENTION

The present disclosure generally relates to an epidemic alert system. Particularly, it relates to an epidemic alert system that use vaccination information of a person.

### BACKGROUND OF THE DISCLOSURE

People receive vaccinations in an effort to avoid becoming ill from an infectious disease and to avoid spreading the infectious disease to others because they failed to become ill. Many people have vaccination records/information that track which vaccinations they have had. Systems exist that monitor the outbreak of diseases in certain locations.

### SUMMARY OF THE DISCLOSURE

According to the present disclosure, a method for providing vaccination information is provided. The method includes the steps of receiving vaccination records about a person, receiving travel information about the person including a destination of travel, receiving infectious outbreak information about the destination, determining vaccination timing information based on the travel information and the vaccination records of the person, and transmitting the vaccination information to the person.

According to another aspect of the present disclosure, another method for providing vaccination information is provided. The method includes the steps of receiving vaccination records about a person, receiving travel information about the person including a destination of travel, using at least one route to the destination to identify at least one intermediate location along the at least one route, receiving infectious outbreak information about the at least one intermediate location, and transmitting vaccination information to the person based on the infectious outbreak information.

According to another aspect of the present disclosure, another method for providing vaccination information is provided. The method includes the steps of receiving vaccination records about a person, receiving travel information about the person including a destination, identifying at least one route to the destination, the at least one route having at least one location along the route, receiving infectious outbreak information within a radius of the at least one location, and transmitting vaccination information to the person based on the infectious outbreak information and the vaccination record.

### BRIEF DESCRIPTION OF THE DRAWINGS

The above mentioned and other features of this disclosure, and the manner of attaining them, will become more apparent and the disclosure itself will be better understood by reference to the following description of embodiments of the disclosure taken in conjunction with the accompanying drawings, where:

FIG. 1 is a block diagram of an epidemic alert system;

FIG. 2 is a block diagram of a part of the epidemic alert system of FIG. 1;

FIG. 3 is a block diagram of another part of the epidemic alert system of FIG. 1; and

FIG. 4 is a flow diagram of an illustrative method for providing vaccination information.

For the purposes of promoting an understanding of the principles of the disclosure, references will be made to the embodiments illustrated in the drawings, which are described below. The embodiments disclosed in the following detailed description are not intended to be exhaustive or limit the disclosure to the precise form disclosed in the following detailed description. Rather, the embodiments are chosen and described so that others skilled in the art may utilize their teachings. Unless otherwise indicated or apparent, the components shown in the figures are proportional to each other. It will be understood that no limitation of the scope of the disclosure is thereby intended. The disclosure includes any alterations and further modifications in the illustrative devices and described methods and further applications of the principles of the disclosure which would normally occur to one skilled in the art to which the disclosure relates.

### DETAILED DESCRIPTION OF THE DRAWINGS

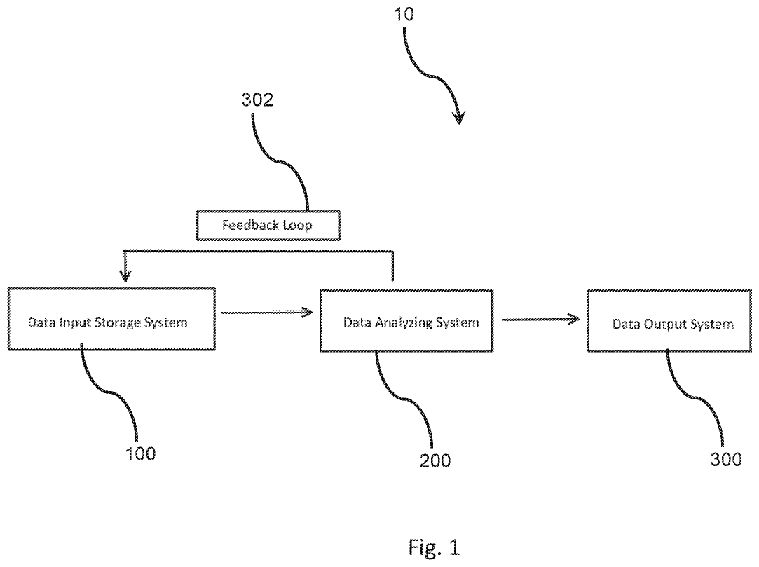

Referring to FIG. 1, an epidemic alert system 10 includes three sub-systems: a data input and storage system 100, a data analyzing system 200, and a data output system 300. In general, data input and storage system 100 receives data from external or internal sources and store the data in at least one data storage device 110 such as a server. Data analyzing system 200 analyzes the data stored in server 110 and sends the analyzed results to data output system 300. Data output system 300 reports the results to a person such as an end user and provides instructions to the person.

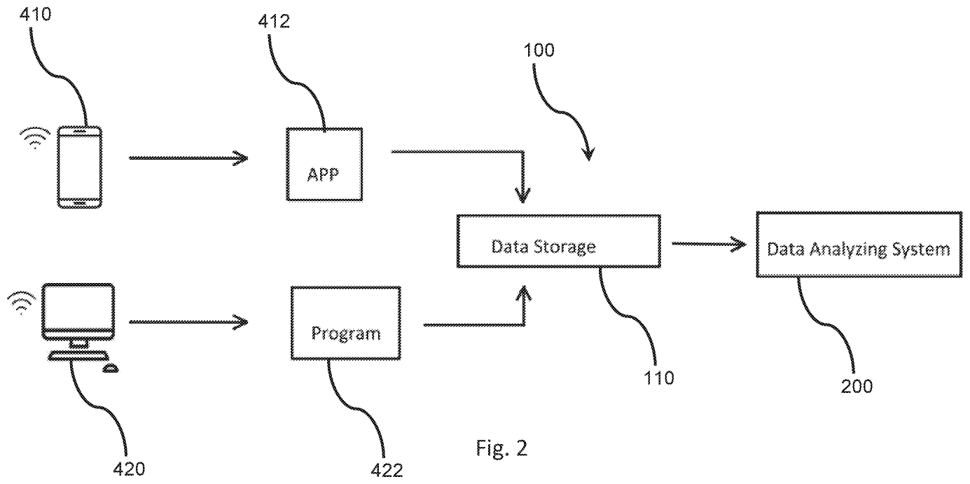

Data input and storage system 100 receives and stores data. As shown in FIG. 2, the data can be from a smartphone 410, a computer 420, or any other electronic device that has an application (app) or a program for data input. The data is transmitted through the Internet or any other communications network to send the data to data storage server 110 of date input and storage system 100. The data in smartphone 410 can be transmitted by control of an app. The data in computer 420 can be transmitted by control of a program.

There are at least three types of information that are received through a smartphone 410 or a computer 420. The first type is the user's personal information, including the user's vaccination records, other medical records, and calendar, which includes travel schedules, destinations and any intermediate locations. A user provides his/her information through smart phone 410 or computer 420 using an app/program provided. The second type of information is the epidemic/medical information provided by local hospitals, medical providers, or other sources of disease information, such as the United States Centers for Disease Control and Prevention, a national health agency of the United States government. Data storage 110 collects and stores such information for further analysis purposes. The third type of information is the internally-generated information created during analysis by data analysis system 200. The internally-generated information assists system 10 in providing more accurate and useful information. Data storage 110 collects and stores internally-generated information as well.

Data analyzing system 200 analyzes the input data and uses a machine learning process that combines the user's personal data and the epidemic/medical data using an algorithm to detect or predict disease outbreaks and create personalized instruction to a user. For example, a user tells the system that he/she will be travelling from Toledo, Ohio to New York City in two weeks with a layover in an intermediate location, such as Indianapolis. System 10 also receives information from Indianapolis or other Indiana hospitals that there has been an outbreak of a disease, such as measles in Indianapolis, as well as New York City's local information indicating that there is a sharp increase of people who have purchased Tamiflu, an antiviral medicine for treatment of flu, or other information indicating an outbreak in New York City of any disease (mumps, measles, Covid-19, etc.). System 10 knows that there is a measles outbreak in Indiana, in particular Indianapolis, and learns that there is or may be a flu outbreak in New York City. As a result, system 10 notifies the user about the measles outbreak in Indiana and the predicted flu outbreak in New York City. If the user indicated that he/she has not received the vaccination for measles or the particular flu virus (or any other identified disease) causing the outbreak in New York City, system 10 will also analyze what vaccine options can be provided to the user to insure proper vaccination prior to the user arriving in the Indianapolis airport and then finally in New York City. System 10 will also analyze and recommend the timing of these vaccines to ensure the user has all necessary vaccinations in time for the travel date, including any necessary vaccinations boosters. Further, system 10 also analyzes an estimated radius of infection based on infectious outbreak information. The radius is the area surrounding an outbreak where the user might be reasonably exposed to the infectious outbreak Based on the estimated radius of infection of an infectious outbreak, system 10 will analyze what vaccine options can be provided to the user to ensure proper vaccination for any overlap the user's travel route has within the radius of infection. Using the same example as above, if system 10 receives information that there is an outbreak of SARS 40 miles from New York City and the estimated infection radius is analyzed to be 70 miles from the outbreak, system 10 will notify the user of the SARS outbreak outside New York City and analyze what vaccine options can be provided to the user to ensure proper vaccination for Indianapolis, New York City, and the nearby infectious outbreaks to those locations. In another example, a user may indicate they are planning to visit a location prone to certain diseases. For example, a resident of the United States may be planning a visit to a certain location in Africa. Because vaccination regimens in the United States do not often include vaccinations that are warranted in the certain location in Africa, the user may be missing certain recommended vaccinations. System 10 will notify the user of the missing vaccinations prior to the user's trip to Africa to provide sufficient time for the user to receive the recommended vaccinations. Further, system 10 will notify the user of any boosters the user might need and the timing of the vaccinations prior to travel. For example, if system 10 identifies a needed vaccination, it may notify the user of the needed vaccination, a deadline by which the vaccination must occur based on the vaccines effectiveness time period (i.e. the time between when a vaccine is administered and when the vaccine become effective against a disease) or a warning against travel if there is insufficient time for

The personal information of the user collected by the smartphone 410 or computer 420 also includes any intermediate locations along the user's travel route that are not specifically indicated on the user's travel itinerary. For example, the user travelling from Toledo, Ohio to New York City, may reside in Ft. Wayne, Ind. and planning on driving to Toledo for a flight. System 10 will collect this information and determine if any actual or potential outbreaks exist along one or more routes to Toledo from the user's residential address. For example, system 10 may recognize an outbreak or potential outbreak in Defiance, Ohio, a likely intermediate location along a potential driving route between the user's residential address and Toledo. System 10 may also be used for shorter and longer routes using various modes of transportation such as airplanes, ground vehicles, ships, trains, etc. and combinations thereof. For example, a user may use system 10 for travel from Ft. Wayne, Ind. to Guangzhou, China using a car from Ft. Wayne to Toledo, Ohio, an airplane from Toledo to Hong Kong with layovers in Indianapolis, Ind., New York City, San Francisco, Calif., a ferry from Hong Kong to Guangzhou, and a train or subway from the ferry to the final destination in Guangzhou, China. System 10 may then analyze all or a portion of the intermediate locations along possible routes between the user's residential address in Ft. Wayne and the final destination in Guangzhou, China. System 10.

System 10 includes a feedback loop 302 (as shown in FIG. 1) between data input and storage system 100 and data analyzing system 200. Feedback loop 302 can further help data analyzing system 200 reach accurate results. For example, if many users tell system 10 that they traveled from place A to place B, and place A just had a flu outbreak, system 10 can predict that place B may also have a flu outbreak soon and feed such information back into data input system 100 to help determine what information should be provided to other users who plan to travel to place B.

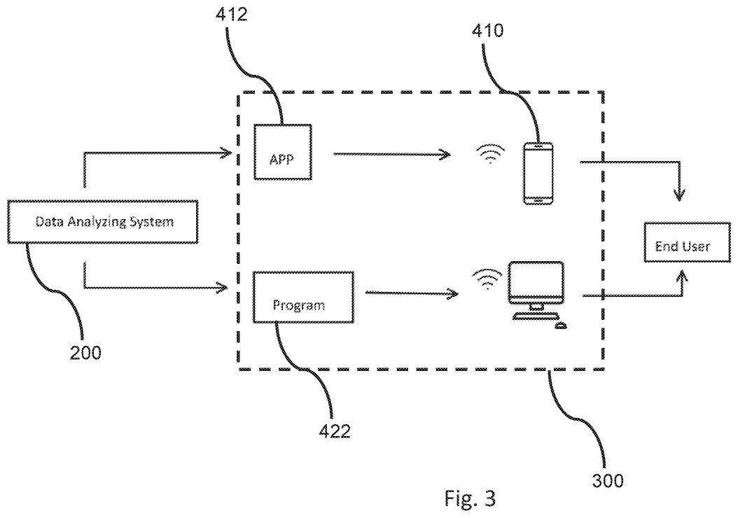

As shown in FIG. 3, data analyzing system 200 sends its results to data output system 300 so the users will be notified. Data output system 300 can include a smartphone 410 equipped with an app, computer 420 equipped with a program, or any other electronic devices that has an application or a program for receiving data and reporting information. Data output system 300 can connect to the Internet to receive information from data analyzing system 200. A user can use the same or a different device to input their information and receive notification. Each user will have his/her own account though which he/she can access the system in any devices that has the application/program to input and read information. A user, such as a family member (or medical care provider or another with access to the information), may also input his/her family's (or patient's, client's, customer's, etc.) information in the account, so system 10 can report information to the user regarding all or other family members (or patients, clients, or customers, etc.).

With the information provided to the user by data output system 300, a user will assess their risk of infectious disease, get the generated recommended vaccinations and respond by updating their vaccinations with recommended injections, etc. of initial vaccinations, boosters, etc. Additionally, the user may potentially change their route of travel to avoid areas with current, potential, or predicated outbreaks. System 10 may provide alternative routes to avoid the need to update vaccinations or the user may propose such routes. The user inputs information about new vaccinations they received and any changes to the travel plan(s). Data analyzing system 200 analyzes the updated personal data to create routes of travel based on the user's current vaccination records, ability to get the recommended vaccination, and travel schedule.

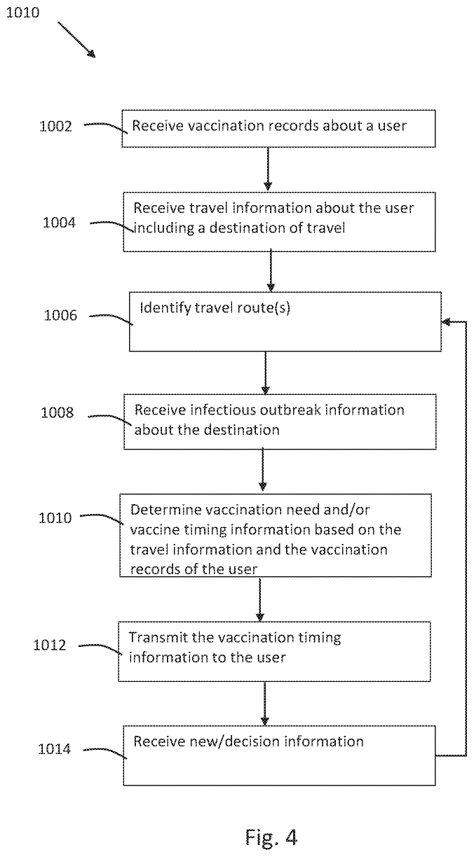

FIG. 4 shows a flow diagram of an illustrative method 1000 for providing vaccination information with epidemic alert system 10. Method 1000 includes a vaccination information receiving step 1002 to receive vaccination records about a user, a travel information receiving step 1004 to receive travel information about the user including a destination of travel, intermediate locations, such as layovers, modes of transportation, departure and return dates, etc., an route identifying step 1006 to identify one or more routes to the destination including intermediate locations along the route(s), an infectious disease information step 1006 to receive infectious outbreak information about the destination, intermediate locations, and locations within a determined radius of the destination and intermediate locations, information about other travelers to these locations from locations with current, potential, or predicated outbreaks, etc., a vaccination need and timing step 1010 to determine need and vaccination timing information based on the travel information (e.g. proposed departure time) and the vaccination records of the user, etc., and vaccine information transmission step 1012 to transmit the vaccination information to the user, etc. As mentioned above, the travel information of the user may contains an intermediate location along a route to the destination. According to an exemplary embodiment, this method may further include steps of receiving infectious outbreak information about the intermediate location, and transmitting vaccination information to the user based on the intermediate location and the vaccination records of the user. The vaccination information may include a needed vaccination of the user. The needed vaccination could be an initial vaccination and/or a booster recommendation. According to an exemplary embodiment, this method may further include a step of receiving access to a calendar of the user, wherein the calendar may include the travel information (departure time, destination, layovers, mode(s) of transportation, flight numbers, etc.).

Method 1000 may further include a decision information receiving step 1014 to receive new/decision information from the person based on the vaccination information transmitted to the person of otherwise. For example, the person may select a suggested alternative route from system 10, chose their own route, changed their travel plans in response to step 1012, provide updated vaccination records, etc. and provide this new information to system 10. System 10 uses this new information in step 1006 to determine new routes, if any, to the destination to identify any additional intermediate locations along an new/additional route based on the new information and infectious outbreak information about the additional intermediate location. The vaccination information may include a plurality of needed vaccination of the user based on infectious outbreak information about the destination and the infectious outbreak information about the intermediate location. According to an exemplary embodiment, the step of using the route may include using a plurality of routes to the destination to identify intermediate locations along the routes and using the plurality of routes. The step of receiving the infectious outbreak information may include receiving infectious outbreak information about intermediate locations. The step of transmitting the vaccination information to the person is based on the infectious outbreak information about the intermediate locations.

According to an exemplary embodiment, the infectious outbreak information may include a distance or radius between a determined location, such as a destination, intermediate location, etc. and a place associated with the infectious outbreak information. The radius is based on where the user might be reasonably expected to travel beyond a determined location and potentially be exposed to an infectious outbreak that is away from the determined location. If an infectious disease outbreak in identified within this radius, system 10 warns the user of needed vaccinations for the identified disease so they user can receive the needed vaccination and/or avoid the location of the identified outbreak. Similarly, system 10 may identify other travelers to the determined location (destination, intermediate location) from areas of current or potential outbreaks. If a sufficient number of other traveler are (or will be) travelling to the determined location from these outbreak areas, system 10 warns the user of needed vaccinations for the identified disease. After using system 10, the user may receive the needed vaccination, delay or cancel their travel plans, alter their travel plans, and/or eventually travel to the destination only a route with potential stops at intermediate locations

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for providing vaccination information comprising steps of
receiving vaccination records about a person,
receiving travel information about the person including a destination of travel,
receiving infectious outbreak information about the destination,
determining vaccination timing information based on the travel information and the vaccination records of the person, and
transmitting the vaccination timing information to the person.

2. The method of claim 1, wherein the travel information contains at least one intermediate location along a route to the destination, further comprising steps of
receiving infectious outbreak information about the at least one intermediate location, and
transmitting vaccination information to the person based on the at least one intermediate location and the vaccination records of the person.

3. The method of claim 1, wherein the vaccination information includes at least one needed vaccination of the person.

4. The method of claim 3, wherein the needed vaccination is a booster recommendation.

5. The method of claim 1, further comprising a step of receiving access to a calendar of the person, the calendar including the travel information.

6. A method for providing vaccination information comprising steps of
receiving vaccination records about a person,
receiving travel information about the person including a destination of travel,
using at least one route to the destination to identify at least one intermediate location along the at least one route,
receiving infectious outbreak information about the at least one intermediate location, and
transmitting vaccination information to the person based on the infectious outbreak information.

7. The method of claim 6, wherein further comprising steps of
receiving decision information from the person based on the vaccination information transmitted to the person, and
using the at least one route to the destination to identify at least one additional intermediate location along at least one additional route based on the decision information and infectious outbreak information about the at least one additional intermediate location.

8. The method of claim 6, wherein the vaccination information includes a plurality of needed vaccination of the person based on infectious outbreak information about the destination and the infectious outbreak information about the at least one intermediate location.

9. The method of claim 6, wherein
the step of using the at least a route includes using a plurality of routes to the destination to identify intermediate locations along the routes and using the plurality of routes,
the step of receiving the infectious outbreak information includes receiving infectious outbreak information about intermediate locations, and
the step of transmitting the vaccination information to the person is based on the infectious outbreak information about the intermediate locations.

10. A method for providing vaccination information comprising steps of
receiving vaccination records about a person,
receiving travel information about the person including a destination,
identifying at least one route to the destination, the at least one route having at least one location along the route,
receiving infectious outbreak information within a radius of the at least one location, and
transmitting vaccination information to the person based on the infectious outbreak information and the vaccination record.

11. The method of claim 10, wherein infectious outbreak information includes a distance between the location and a place associated with the infectious outbreak information.

12. The method of claim 10, wherein the radius is based on where the person might be reasonably exposed to an infectious outbreak.

13. The method of claim 10, wherein the vaccination information is based on an estimated infection radius overlapping with radius of the at least one location.

14. The method of claim 10, wherein the vaccination, wherein the vaccination information includes at least one needed vaccination of the person.

15. The method of claim 10, further comprising a step of transmitting at least one vaccination timing information to the person based on the travel information and the vaccination records of the person.

16. The method of claim 10, wherein the infectious outbreak information is received from at least one of local hospital, a medical provider, and a national health agency.
